(19) United States
(12) Patent Application Publication
(10) Pub. No.: US 2010/0184031 A1
(43) Pub. Date: Jul. 22, 2010

(54) NOVEL ADIPOCYTOKINE VISFATIN/PBEF1 IS AN APOPTOSIS ASSOCIATED FACTOR INDUCED IN MONOCYTES DURING IN VIVO HIV-1 INFECTION

(76) Inventors: Geert Raes, Sint-Genesius-Rode (BE); Patrick De Baetselier, Berchem (BE); Rafael Van den Bergh, Berchem (BE); Guido Vanham, Lier (BE)

Correspondence Address: TRASKBRITT, P.C., P.O. BOX 2550, SALT LAKE CITY, UT, 84110, US

(21) Appl. No.: 12/451,864

(22) PCT Filed: Jun. 5, 2008
(86) PCT No.: PCT/EP2008/057025

§ 371 Date: Mar. 18, 2010

Related U.S. Application Data
(60) Provisional application No. 60/933,774, filed on Jun. 8, 2007.

(51) Int. Cl. C12Q 1/68

(52) U.S. Cl. 435/6

(57) ABSTRACT

The present invention relates to the use of monocyte markers for diagnostic, prognostic or theranostic applications during diseases and syndromes caused by HIV infection. More specifically, it relates to a method comprising isolation of monocytes and determining gene expression, preferably PBEF1 gene expression. The method is useful to determine the evolution of the disease or can be used to evaluate the efficacy of a treatment.

### CROSS-REFERENCE TO RELATED APPLICATION

This is a national phase entry under 35 U.S.C. §371 of International Patent Application PCT/EP2008/057025, filed Jun. 5, 2008, published in English as International Patent Publication WO 2008/148858 A1 on Dec. 11, 2008, which claims the benefit under Article 8 of the Patent Cooperation Treaty and 35 U.S.C. §119 to U.S. Provisional Patent Application Ser. No. 60/933,774, filed Jun. 8, 2007.

### TECHNICAL FIELD

The present invention relates to the use of monocyte markers for diagnostic, prognostic or theranostic applications during diseases and syndromes caused by HIV infection. More specifically, it relates to a method comprising isolation of monocytes and determining gene expression, preferably PBEF1 gene expression. The method is useful to determine the evolution of the disease or can be used to evaluate the efficacy of a treatment.

### BACKGROUND

Monocytes and macrophages, as their more differentiated counterparts, play a fundamental role during HIV infection since they act as both antigen-presenting cells and effector cells of cellular immunity. While monocytes can be infected by HIV, they do not enter apoptosis upon HIV infection. Hence, they can act as a reservoir for the virus where it can continue to replicate even during Highly Active Antiviral Therapy (HAART) (reviewed in, e.g., Aquaro et al., 2002). Infected monocytes display dysfunctional behavior concerning the elimination of pathogens, which gives rise to a number of opportunistic infections (e.g., Kedzierska et al., 2003). Additionally, they are capable of recruiting uninfected lymphocytes to sites of infection and rendering these cells susceptible for HIV infection in these cells (Swingler et al., 1999; Swingler et al., 2003). Furthermore, they have been shown to induce apoptosis in uninfected CD4⁺ and CD8⁺ T lymphocytes by a complex interplay of lymphocyte activation, membrane cross-linking (via the CD4 and CXCR4 receptors) and activation of apoptotic pathways, reviewed in Mahlknecht & Herbein, 2001. Conversely, they confer a form of protection against apoptosis on infected CD4⁺ T lymphocytes (Mahlknecht & Herbein, 2001). In the context of HIV-associated disorders, inappropriate monocyte/macrophage activation in the central nervous system (CNS) leading to neuronal apoptosis, is the primary cause of HIV-associated dementia (HAD) (Anderson et al., 2002), and we recently formulated the hypothesis that monocyte/macrophage hyperactivation during HIV infection could be involved in the novel Immune Reconstitution Disease (IRD) (Van den Bergh et al., 2006).

In this study, we analyzed the molecular basis of these monocyte/macrophage dysfunctions using, amongst others, microarray technology. In this fashion, we aimed on the one hand to gain fundamental insight in monocyte/macrophage biology during HIV infection and, on the other hand, to identify potential predictors/molecular markers of HIV- or HAART-associated disorders (such as HAD, IRD and lipodystrophy). Using both a commercially available genome-wide microarray platform and a custom designed “Macrophage Activation State” (MAS) cDNA array containing 700 fragments of genes of interest, we identified monocyte gene expression patterns associated with in vivo HIV infection. We found that in vivo HIV-1 infection induces aberrant gene expression profiles within the circulating monocyte population, and that these gene expression patterns are sufficient to make a distinction between HIV-1-seropositive and -seronegative blood donors. Subsets of these differentially regulated genes can be clustered together in common pathways/processes. Through over-representational analysis, we identified clusters of apoptosis-associated and lipid metabolism/insulin signaling-related genes, which appear to be disrupted by HIV infection. Unexpectedly, the adipocytokine visfatin (also known as pre-B-cell colony-enhancing factor 1 or PBEF1 or nicotinamide phosphoribosyltransferase or NAMPT) was found to be one of the genes of which the expression is induced in monocytes of HIV patients. PBEF1 expression correlated with the plasma viral load (rather than the CD4+ lymphocyte count) in these patients, suggesting that the presence of virus by itself can be responsible for changes in monocyte phenotype, rather than secondary effects mediated through dysfunction of the T lymphocyte population. As the infectivity of HIV for monocytes is limited, it is unlikely that PBEF1 induction is a result of direct infection of the cell. Moreover, our in vitro experiments have shown that treatment of monocytes from non-infected individuals with either infective or AT2-inactivated HIV_{BaL} virus induces PBEF expression, possibly by the triggering of receptors on the monocyte membrane by intact virus or individual viral components, which was demonstrated previously to be sufficient to induce changes in monocyte/macrophage phenotype (e.g., Freedman et al., 2003). We also found that PBEF1 expression was reduced again upon HAART, associated with the reduction in viral load.

PBEF1 is a relatively novel, illusive cytokine/adipokine (Samal et al., 1994), which has rapidly been gaining interest the past years, especially in the context of obesitas and diabetes research as a result of its insulin-mimetic properties (reviewed in Stephens & Vidal-Puig, 2006). It is induced by inflammatory cytokines in epithelial cells (Ognjanovic et al., 2005) and leukocytes (Jia et al., 2004). In turn, it activates leukocytes and induces, amongst others, IL-1β, TNF-α and IL-6 in monocytes (Moschen et al., 2007). Additionally, it is involved in intracellular regulation of nicotinamide adenine dinucleotide (NAD⁺)-dependent reactions (van der Veer et al., 2005). Schindler et al. (2006) recently described increased plasma levels of PBEF1 in HIV patients on HAART, but not in therapy-naïve HIV patients. They propose the interesting hypothesis that the increase of PBEF1 serves to compensate for HAART-induced insulin resistance. Our observation that PBEF1 expression in patients on HAART was reduced to the levels also found in healthy controls, suggests that circulating monocytes play no role in an increase in plasma PBEF1 upon HAART as described to occur by Schindler et al. On the other hand, our finding that expression of the insulin-mimetic (and insulin-sensitizing) PBEF1 is perturbed in the pre-therapy stage in circulating monocytes, provides a further suggestion that insulin resistance (and the associated metabolic syndrome) are heralded by pro-inflammatory events before therapy is initiated (e.g. Aboud et al., 2007).

Of further interest, PBEF1 exerts an anti-apoptotic effect on neutrophils during inflammation and sepsis (Jia et al., 2004), as well as on amniotic epithelial cells and fibroblasts (Ognjanovic et al., 2005). As monocyte dysfunction during HIV infection is characterized by a persistent failure to enter apoptosis, as well as by anti-apoptotic effects mediated on HIV-infected T lymphocytes, PBEF1 may well be an important factor in this dysfunction. The immunogenic properties of PBEF1, on the other hand, and especially its activating properties on myeloid cells (Moschen et al., 2007), suggest an involvement in the recruitment of, and induction of viral production in, host T lymphocytes or a controlling function on HIV latency in cells of myeloid lineage.

### DISCLOSURE OF THE INVENTION

Considering the expression of PBEF1 and the other genes disclosed in Tables 4 and 5 in monocytes of HIV-infected patients, these genes and their gene products represent monocyte markers of value for diagnostic, prognostic or theragnostic applications during HIV infection. Moreover, considering the possible involvement of PBEF1 in monocyte dysfunction during HIV infection, PBEF1 represents a therapeutic target in monocytes/macrophages during HIV infection. PBEF1 genes have been described in humans (GenBank accession number NM_{—}005746 and NP_{—}005737), mice (GenBank accession number NM_{—}021524 and NP_{—}067499) and rats (GenBank accession number NM_{—}177928 and NP_{—}808789). The GenBank numbers are cited as non-limiting examples of PBEF1 genes.

A first aspect of the invention is the use of the marker gene expression level for diagnosis, prognosis or theranosis of disease. Preferably, the use is for theranosis. Preferably, the disease is a disease caused by HIV infection. More preferably, the disease is a disease caused by HIV-1 infection. More preferably, the disease is selected from the group consisting of Acquired Immune Deficiency Syndrome (AIDS) or the HIV- or HAART-associated disorders HIV-associated dementia (HAD), Immune Reconstitution Disease (IRD) and lipodystrophy. Preferably, the marker is a gene selected from the genes mentioned in Table 4. Even more preferably, the marker gene is a gene selected from the genes mentioned in Table 5. More preferably, the marker is a gene mentioned in Table 5. Most preferably, the marker is PBEF1.

Methods to measure the expression level of the marker are known to the person skilled in the art and include, but are not limited to, DNA-RNA hybridization and PCR-related methods, using primers specific for the marker messenger RNA. Alternatively, the expression level may be measured at the level of the protein, using, as a non-limiting example, antibody-based techniques such as ELISA. Still another way to measure the expression level is by the use of a reporter gene, operably linked to the marker promoter. “Operably linked” refers to a juxtaposition wherein the components so described are in a relationship permitting them to function in their intended manner. A promoter sequence operably linked to a coding sequence is ligated in such a way that expression of the coding sequence is achieved under conditions compatible with the promoter sequence. Alternatively, the reporter gene is fused to a coding sequence of the marker and expressed as a fusion protein, comprising a part of the marker amino acid sequence up to the total sequence. Suitable reporter genes are known to the person skilled in the art and include, but are not limited to, antibiotic resistance genes, genes encoding fluorescent proteins, or genes encoding surface markers.

Diagnosis or theranosis of the monocyte population can help to identify and treat the disease. “Theranosis” as used herein is a diagnostic method, wherein the results are used to follow the evolution of the disease, to evaluate the efficacy of the medication and/or to adapt the treatment in function of the result of the diagnosis. Following the evolution of the monocyte population during the treatment, the marker allows theranosis in those diseases where there is an imbalance in macrophage/monocyte populations.

Another aspect of the invention is a method for diagnosis, prognosis or theranosis of HIV-related diseases, comprising (a) collection of a blood sample from a subject, (b) isolation of the monocytes from this blood sample and (c) determination of gene expression in the monocytes. Preferably, “expression” is the expression of a marker gene selected from the list of Table 5. Even more preferably, “expression” is the expression of a marker gene selected from the group consisting of ADORA1, PBEF1, TNFAIP3, STAT1 (α), STAT1 (β), DDIT3 and BNIP2 (Table 4). Most preferably, “expression” is the expression of PBEF1 mRNA or the detection of its gene product. HIV-related diseases as mentioned herein can be any HIV-related disease. Preferably, the disease is selected from the group consisting of Acquired Immune Deficiency Syndrome (AIDS) or the HIV- or HAART-associated disorders HIV-associated dementia (HAD), Immune Reconstitution Disease (IRD) and lipodystrophy.

Preferably, expression of PBEF1 or the detection of the gene product is used as a marker for detection of co-receptor usage and/or co-receptor switch. The biomarker can be used to follow the switch from moderately virulent viruses to more aggressive strains, and is useful both in theranosis and in follow up of the effect of the treatment.

Another aspect of the invention is the use of PBEF1 as target for therapy aimed at repression or reduction of disease. Preferably, the target of therapy is PBEF1 expressed in myeloid cells.

More preferably, the target of therapy is PBEF1 expressed in macrophages or monocytes. Even more preferably, the target of therapy is PBEF1 expressed in monocytes. Preferably, the disease is a disease caused by HIV infection. More preferably, the disease is a disease caused by HIV-1 infection. Most preferably, the disease is selected from the group consisting of Acquired Immune Deficiency Syndrome (AIDS) or the HIV- or HAART-associated disorders HIV-associated dementia (HAD), Immune Reconstitution Disease (IRD) and lipodystrophy. Repression or reduction of the disease can be realized either by limiting the expression of PBEF1 in the cells or by impairing or inhibiting the binding capacity or enzymatic activity of PBEF1 in the cells. Limitation of expression can be obtained, as a non-limiting example, by inactivating the PBEF1-encoding gene in the cells, by inactivation of the promoter of the PBEF1-encoding gene in the cells, or by expressing PBEF1 RNAi in the cells. Methods to impair or inhibit the binding capacity or enzymatic activity of PBEF1 in the cells are known to the person skilled in the art and include, but are not limited to, the targeting of anti-PBEF1 antibodies, anti-PBEF1 antibody fragments, or inhibitors of PBEF1 enzyme activity to the cells. Antibodies and antibody fragments as used here include, but are not limited to, classical antibodies, single chain antibodies, camelid antibodies and nanobodies. Specific inhibitors of PBEF1 enzymatic activity have been described and include, but are not limited to, the anti-cancer agent FK866. Methods for targeting the antibodies, antibody fragments, or inhibitors to the cells are known to the person skilled in the art and include, but are not limited to, chemical or genetic coupling of the antibodies, antibody fragments or inhibitors to antibodies, or antibody fragments recognizing surface markers on the cells.

### BRIEF DESCRIPTION OF THE DRAWINGS

FIG. 1. Principal Components Analysis (PCA) on “present” mean-normalized CodeLink datasets of HIV patient and healthy control samples. HIV patient samples are represented in light grey, healthy control samples in dark grey: a clear distinction can be made between the cluster of HIV patient samples and the healthy controls.

FIG. 2. Expression values of PBEF1, as assessed by CodeLink HWG analysis, after mean normalization of the dataset (Panel A) and by the Macrophage Activation State (MAS) array (Panel B). Expression of PBEF1 in HIV patient samples is significantly higher than in healthy control samples (uncorrected t-test, p=0.001).

FIG. 3. Panel A) Expression of PBEF1, normalized to GAPDH expression, as assessed by RT-QPCR and plotted versus CD4+ T lymphocyte count. Up-regulation is significant in patients with 200<T4<500 cells/mm<3> (Mann-Whitney, p<0.05). Panel B) Expression of PBEF1, normalized to GAPDH expression, as assessed by RT-QPCR and plotted versus viral load. A positive correlation between viral load and PBEF1 expression was seen (r²=0.4881; p=0.001).

FIG. 4. Expression of PBEF1, normalized to GAPDH expression, as assessed by RT-QPCR. PBEF1 is significantly up-regulated in therapy-naïve HIV patients, but not in patients on HAART (Mann-Whitney, p<0.05).

FIG. 5. Expression of PBEF1 in elutriation-purified monocytes after stimulation with mock- and AT2-inactivated HIV_{BaL}, normalized to GAPDH expression and expressed relative to non-treated controls.

FIG. 6. Productive infection of PBMC cultures by HIV_{BaL}, as quantified by p24 secretion detected by ELISA, in presence and absence of visfatin.

FIG. 7. Viral infectivity, expressed as TCID50, in monocyte-derived macrophages (Panel A) and PBMC (Panel B) after 14 days of culture in presence and absence of 200 ng/ml PBEF1. Representative results of three independent experiments shown.

FIG. 8. Viral infectivity, expressed as TCID50, in PBMC after 14 days of culture in presence and absence of 100 μM nicotinamide mononucleotide.

FIG. 9. Visfatin protein expression in monocytes of HIV patients (as assessed by ECL-Western Blot and normalized to β-actin expression), for patients with R5 or X4 virus (as assayed using infection of viral isolates in CCR5- or CXCR4-expressing U87 cells).

### DETAILED DESCRIPTION OF THE INVENTION

### Examples

### Materials and Methods to the Examples

### Sample Collection

Fifty ml blood samples were collected in EDTA-tubes from therapy-naïve HIV-1-seropositive patients from the HIV Clinic of the Institute of Tropical Medicine in Antwerp, Belgium. Patient details are shown in Tables 1 and 6. Peripheral blood mononuclear cells (PBMCs) were separated via a Ficoll gradient and plasma was concomitantly aspirated and stored at −80° C.

Monocytes were purified from the PBMC fraction using the negative selection-based Monocyte Isolation Kit II from Miltenyi-Biotec (Bergisch Gladbach, Germany), according to the manufacturer's instructions. Yields were minimally 5 million monocytes with a purity>85%, as verified through flow cytometry.

### In Vitro HIV Treatment Experiments

HIV_{BaL} was either inactivated with aldrithiol-2 (AT-2, 200 μM in DMSO at 37° C. for one hour) or mock-inactivated with DMSO alone. Virus was subsequently enriched by filtration over a 100 kDa cut-off membrane, aliquotted and stored at −80° C. until use. Monocytes were purified via counterflow elutriation and subsequent E-rosetting from buffy coats of healthy blood donors from the Blood Transfusion Centre of Antwerp. Cells were cultured at 3×10<6> cells/ml in RPMI medium supplemented with 10% fetal bovine serum and were treated with infectious and AT-2-inactivated HIV_{BaL} for the indicated times at a concentration corresponding to 50 ng/ml of p24.

### RNA Isolation

For RNA extraction, monocytes isolated from patients or treated in vitro with virus were immediately lysed in Trizol (Invitrogen, Carlsbad, Calif., USA), and Trizol pellets were stored at −80° C. Total RNA was prepared from the Trizol pellets by chloroform extraction, as per the manufacturer's recommendations. Ten randomly selected samples were checked for integrity on a BioAnalyzer (BioRad, Hercules, Calif., USA). No protein contamination or degradation of RNA was detected.

### CodeLink Arrays

Selected RNA samples were prepared and hybridized to CodeLink HWG bioarrays according to the manufacturer's instructions (Amersham Biosciences, Freiberg, Germany). CodeLink datasets were analyzed using the GeneMaths XT software package (Applied Maths, St. Martens-Latem, Belgium). After background correction, genes that were called as “absent” in more than four arrays were eliminated from the datasets. Subsequently, array normalization was performed; both quantile and simple mean normalization were performed, without significant differences in the datasets. In this fashion, a normalized dataset containing only genes with a present call in a minimum of eight arrays was constructed.

Normalized “present” datasets were further analyzed for over-representation of specific processes/pathways. For this type of analysis, two different software applications were used: the freeware program GenMAPP/MAPPFinder (Doniger et al., 2003; on the world-wide web at genmapp.org) and the commercially available package GeneGo (on the world-wide web at genego.com). GenMAPP/MAPPFinder clusters genes together in common pathways/processes using the associated Gene Ontology (GO; Ashburner et al., 2000) annotations; additionally, users can contribute pathways (MAPPs) for which over-representation can also be assessed. GeneGo uses a system of manually curated pathways, which are publicly available on the world-wide web at invitrogen.com/ipath.

### Macrophage Activation State Arrays

The Macrophage Activation State (MAS) array was developed as a focused and flexible tool for the analysis of gene expression patterns in monocytes/macrophages. A collection of 700 genes associated with different macrophage activation states was compiled, using a combination of literature data-mining and human “translation” of murine models of macrophage activation available in our laboratory. Subsequently, gene-specific primers were designed for the genes in this collection and fragments were amplified from total cDNA pools of monocytes under various in vitro and in vivo conditions. These fragments were applied in duplicate on 7×10 cm nylon membranes and were cross-linked to the membranes using UV-exposure.

RNA samples from all patients were selected for analysis on this MAS array. A reverse transcription was performed on 1 μg total RNA using oligo-dT and Superscript II reverse transcriptase (Invitrogen) in the presence of <33>P-dCTP (Amersham Biosciences), and the labeled cDNA was then hybridized to the membranes for 20 hours at 42° C. in NorthernMax hybridization buffer (Ambion, Austin, Tex., USA). Membranes were subsequently washed with SDS-containing buffer at 68° C. and were exposed to a phosphorscreen to reveal bound radioactivity. Phosphorscreens were then scanned in a phospho-imager (BioRad). Spot recognition and quantification, background correction and array normalization were all performed using custom-designed software based on the program ImageJ (Image Processing and Analysis in Java, Sun Microsystems, Santa Clara, Calif., USA).

### Real-Time Semi-Quantitative PCR

Expression of individual genes was examined using real-time semi-quantitative PCR (RTQPCR). cDNA was prepared from 1 μg total RNA using oligo-dT and Superscript II reverse transcriptase (Invitrogen) and gene-specific primers for the gene of interest (PBEF1) and a housekeeping gene (GAPDH) were designed:

| | |
|---|---|
| PBEF1.F: 5′-GGCAAGGTGACAAAAAGCTA-3′ | (SEQ ID NO: 1) |
| PBEF1.R: 5′-ATGAAAGGGCAGTATGTCCA-3′ | (SEQ ID NO: 2) |
| GAPDH.F: 5′-AGCTCATTTCCTGGTATGACA-3′ | (SEQ ID NO: 3) |
| GAPDH.R: 5′-TGGTTGAGCACAGGGTACTT-3′ | (SEQ ID NO: 4) |

PCR reactions were performed in duplicate in a BioRad MyCycler, with BioRad iQ SYBR Green Supermix; each PCR cycle consisted of 60-second denaturation at 94° C., 45-second annealing at 55° C., and 60-second extension at 72° C. Gene expression was normalized using the gene GAPDH, coding for the enzyme glyceraldehyde-3-phosphate dehydrogenase, as a housekeeping gene.

### In Vitro Infection Experiments

For in vitro infection experiments, monocytes were obtained from buffy coats of healthy donors of the Blood Transfusion Center of Antwerp (Rode Kruis Vlaanderen, Belgium) by counterflow elutriation, as described previously (Van Herrewege et al., 2002). These cells were then differentiated to monocyte-derived macrophages (MDM) during seven days in RPMI 1640 medium (Bio-Whittaker, Verviers, Belgium) supplemented with 10% bovine fetal calf serum (Biochrom, Berlin, Germany), penicillin (100 U/ml) and streptomycin (100 μg/ml) (Roche Diagnostics, Mannheim, Germany) and 40 ng/ml M-CSF (PeproTech, London, United Kingdom) at 37° C. and 5.0% CO₂. Half the medium was replaced after four days. Cells were then harvested and used for experiments in the same medium (without M-CSF).

Recombinant visfatin was obtained from PeproTech and Alexis (Zandhoven, Belgium). As both batches gave similar results in preliminary studies, all further experiments were performed using recombinant protein from PeproTech. The recombinant protein batches contained <0.01 ng/μg LPS, as assessed by quantitative chromogenic limulus amoebocyte lysate assay (QLAL) (Bio-Whittaker).

For infection experiments, MDM were plated in 96-well plates at 7.5×10⁵ cells/ml and pre-treated with recombinant visfatin (200 ng/ml) for 1 hour at 37° C. and 5.0% CO₂. Then, virus was added in six-fold and incubated for 2 hours, again at 37° C. and 5.0% CO₂. Cells were then washed 3× to remove unbound virus and incubated for 14 days. Productive infection was monitored via an in-house-developed p24 antigen ELISA, as described elsewhere (Beirnaert et al., 1998).

### Viral Isolation and Co-Receptor Usage Determination

Plasma separated from patient blood samples by Lymphoprep separation was stored at −80° C. until use. One ml samples were added to 5×10⁶ phytohemagglutinin (PHA)/interleukin-2 (IL2) stimulated PBMCs obtained from buffy coats of healthy donors of the Blood Transfusion Center of Antwerp and were cultured in RPMI 1640 medium (Bio-Whittaker) supplemented with 10% bovine fetal calf serum (Biochrom), penicillin (100 U/ml) and streptomycin (100 μg/ml) (Roche), PHA (0.5 μg/ml) (Murex Biotech Ltd., Dartford, United Kingdom) and IL2 (5 ng/ml) (Roche). Medium was refreshed twice weekly, and supernatants were monitored using p24 antigen ELISA. Additional PBMC were added on an ad hoc basis when cells became depleted. Cultures were followed until p24 levels in the supernatants reached overflow values in ELISA. Then viruses were harvested, aliquoted and stored at −80° C.

For co-receptor usage-determination assays, plasma viruses were serially diluted and added to U87.R5 or U87.X4 cells in quadruplicate. Unbound virus was washed away after 2 hours incubation at 37° C. and 5.0% CO₂, and productive infection was monitored by p24 ELISA after 7 and 14 days. Productive infection of either U87.CCR5 or U87.CXCR4, signifying, respectively, R5 and X4 usage, was clear-cut in all cases. HIV_{BaL} and HIV_{IIIB} viruses were assayed in parallel as positive controls for, respectively, R5 and X4 virus.

### Statistical Analysis

For CodeLink HWG bioarray data, an uncorrected t-test (p-value<0.01 significant) and a Benjamini-Hochberg-corrected t-test (p-value<0.05 significant) to control the false positive rate (Benjamini & Hochberg, 1995) were used; for the MAS data, only an uncorrected t-test (p-value<0.05 significant) was used. Significance of RT-QPCR data was assessed via a nonparametric Mann-Whitney test.

### Example 1

### CodeLink Array Hybridizations of Monocyte Samples from Therapy-Naïve HIV-Infected Patients

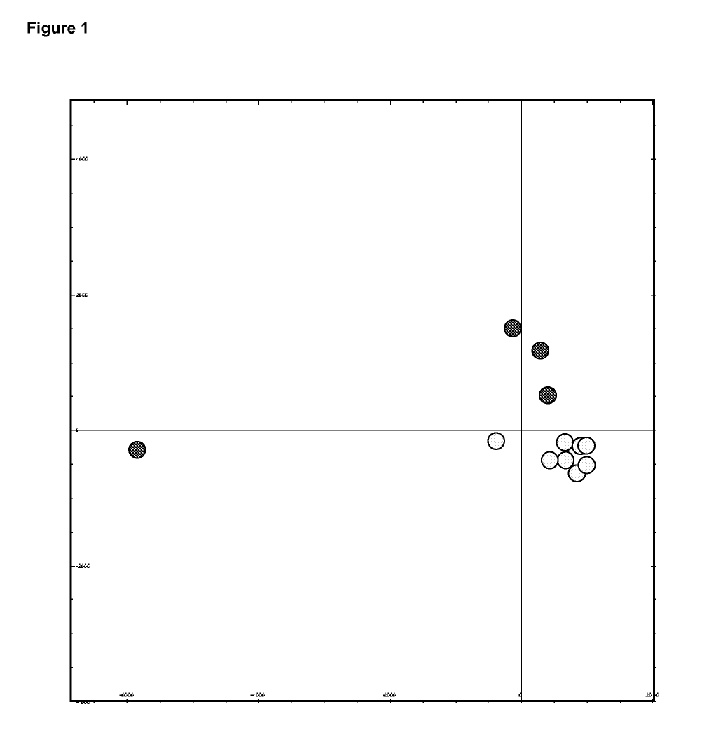

Eight HIV patient samples with a broad range of CD4⁺ T lymphocyte counts and four healthy control samples were selected for analysis on CodeLink HWG microarrays (P01-P08 and C01-C04; Table 1) and were processed as described. A Principal Components Analysis (PCA) was performed on the normalized datasets of “present” genes. This allowed a segmentation of the data into a cluster of HIV-samples, a cluster of control samples and one outlier control sample (FIG. 1), suggesting that monocyte function is distinctly modulated during in vivo HIV infection.

Samples were grouped according to serostatus, i.e., no stratifications according to CD4⁺ T lymphocyte count or viral load were performed, and gene expression values were compared between the HIV-positive and HIV-negative groups. Two different types of meaningful information can be extracted from datasets of this magnitude. On the one hand, by performing over-representational analysis on a broad group of genes for which expression is significantly different (i.e., p-value<0.01 as only criterion), it is possible to identify specific pathways and/or functional groups of genes that are influenced as a whole. On the other hand, by using more stringent criteria (i.e., p-value<0.01 and fold change>1.5), individual genes that may play pivotal roles in the model at hand or that may be candidate molecular markers for certain conditions can be identified.

In the context of this study, over-representational analysis using the two described software applications (GeneGo and GenMAPP) revealed several cellular pathways/processes involved in apoptosis that were significantly modulated in monocytes of HIV patients (Table 2), confirming our notion that the cellular apoptotic machinery is disturbed on a molecular level during HIV infection. Another class of processes that appears to be targeted in monocytes by HIV infection is a group of pathways involved in lipid metabolism and/or insulin signaling (Table 3). To the best of our knowledge, no study to date has focused on these processes in monocytes during HIV infection.

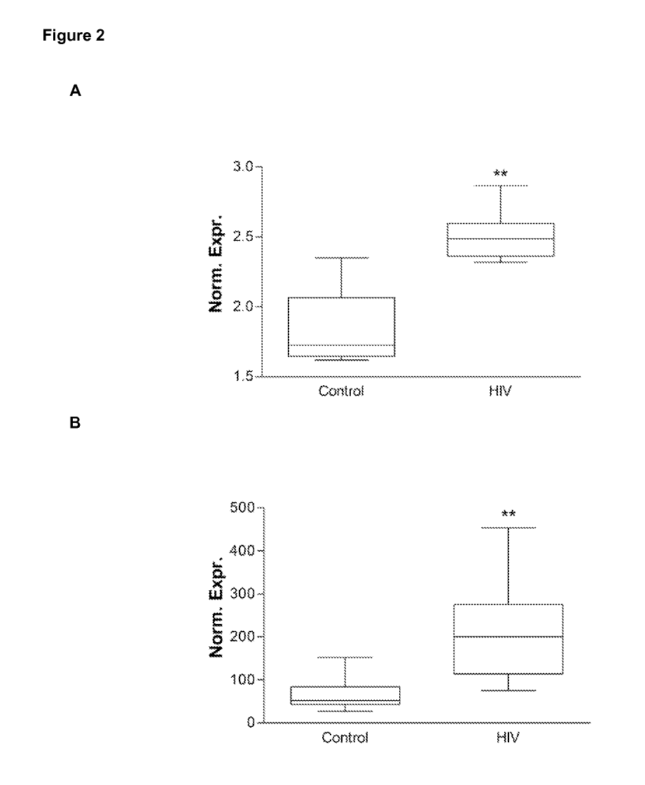

An analysis of the datasets aimed at the identification of individual genes with an interesting expression pattern yields several candidates. One of the genes that pass the set criteria (p-value<0.01, fold change>1.5) is the novel cytokine/adipokine PBEF1 (pre-B-cell colony-enhancing factor 1 or visfatin) (FIG. 2), which possesses a documented involvement in both lipid metabolism and apoptosis.

### Example 2

### MAS Array Hybridizations of Monocyte Samples from Therapy-Naïve HIV-Infected Patients

All HIV patient (n=29) and healthy control (n=8) samples (Table 1) were analyzed on our custom MAS array as described. Patients were grouped together according to their CD4⁺ T lymphocyte count: T4<200 cells/mm<3> (group 1), 200<T4<500 cells/mm<3> (group 2) and T4>500 cells/mm<3> (group 3). As this smaller-scaled array is less geared towards pathway analyses and more towards identification of individual genes of interest, over-representation/pathway analysis was not performed on this dataset. Statistical analysis, however, again with an additional fold change cut-off of 1.5, revealed a list of genes to be significantly up-regulated or to be significantly down-regulated in at least one of these patient groups (Table 4). Within this list, a cluster of apoptosis-associated genes (n=6) could be compiled (Table 5), using the Gene Ontology (GO) annotations in combination with a thorough screening of the available literature.

Several of these genes possess previously documented HIV-associated properties in cells of monocyte lineage. An example is STAT1, which was previously found to be induced in monocytes and monocyte-derived macrophages by in vitro treatment with HIV-1 Nef or infectious HIV (Federico et al., 2001) and in immature dendritic cells by HIV-1 Tat expression or in vitro HIV infection (Izmailova et al., 2003). Additionally, as an interferon-γ-associated transcription factor, STAT1 is involved in many inflammatory pathways and has been implicated in HIV-associated pathogenesis in a multitude of studies (e.g., Abbate et al., 2000; Asensio et al., 2001; Roberts et al., 2003). PBEF1, on the other hand, was, to the best of our knowledge, never associated with monocyte dysfunction during HIV infection and was only very recently linked with HIV infection in general, in the context of HAART-treated HIV patients (Schindler et al., 2006).

### Example 3

### RT-QPCR Verification of PBEF1 Expression in Monocyte Samples from HIV-Infected Patients Versus Healthy Controls

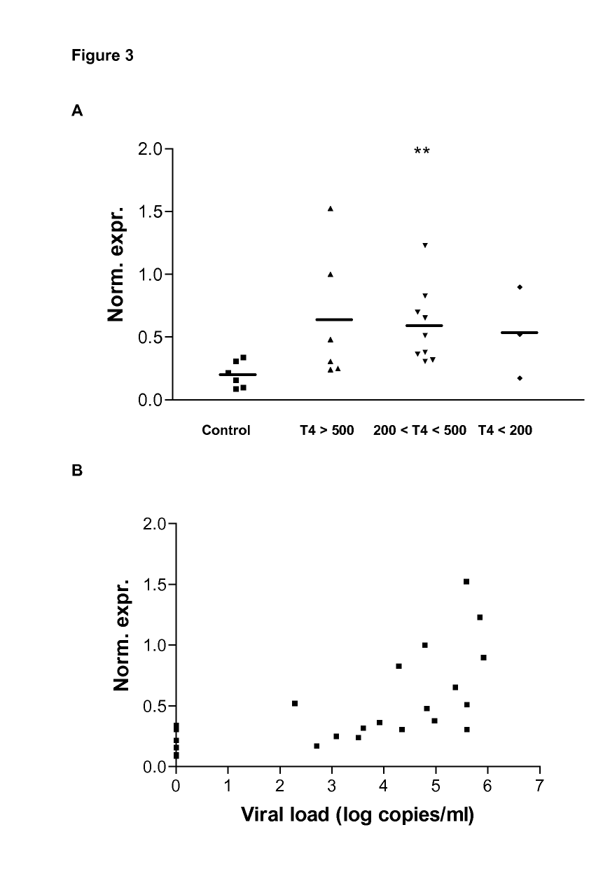

Gene expression was analyzed in selected patient samples (P03-P19 and C01-C06; Table 1) using gene-specific primers for PBEF1. Gene expression data were normalized using the housekeeping gene GAPDH and were compared between HIV patients and healthy controls (FIG. 3). When patients were stratified according to CD4⁺ T lymphocyte count, only group 2 (200<T4<500 cells/mm<3>) appeared to display a significant (Mann-Whitney, p<0.05) up-regulation of PBEF1 (FIG. 3, Panel A), and no significant correlation was found between lymphocyte counts and PBEF1 expression levels. However, according to the lymphocyte counts, this grouping of patients may not be the best strategy, as the aberrant gene expression profile in monocytes may, in the first place, be a direct result of the circulating virus, and correlation between viral load and CD4⁺ T lymphocyte count is not always strong. Therefore, the gene expression was also plotted versus the viral loads of the patients (FIG. 3, Panel B). Linear regression analysis reveals that PBEF1 expression is significantly correlated with the viral load (p=0.001).

### Example 4

### RT-QPCR Analysis of the Effect of HAART on PBEF1 Expression in Monocyte Samples from HIV-Infected Patients

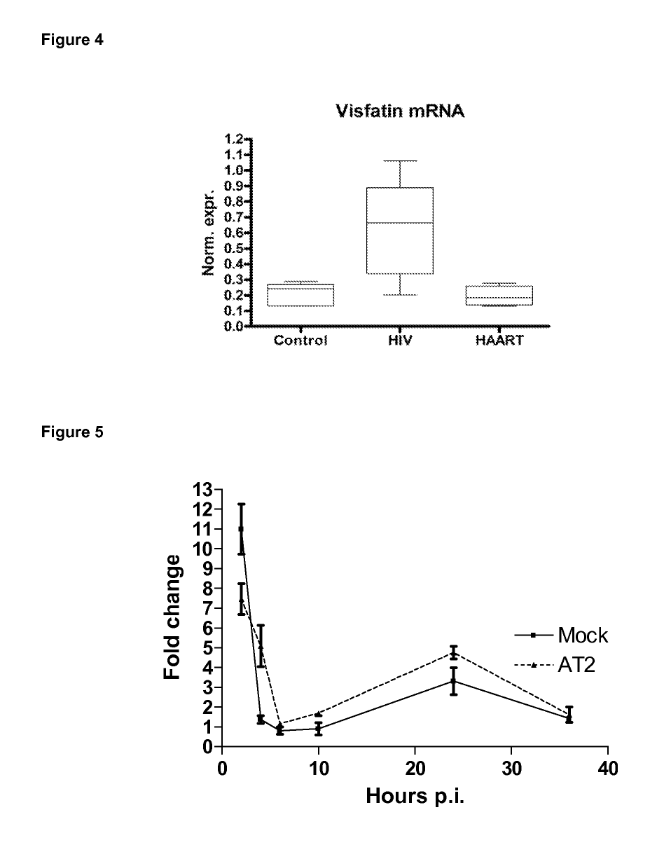

Blood samples were collected from therapy-naïve and HAART-treated HIV patients (Table 6). Body mass index was between 20 and 25 for all patients. Inclusion criteria for therapy-naïve patients were never to have received therapy and to have a viral load of more than four log copies/ml. For HAART patients, inclusion criteria were to have been on therapy for at least one year and to have an undetectable viral load. Samples from healthy seronegative donors with matching age and nationality were collected as negative controls. Monocytes were isolated from these blood samples and PBEF1 expression was analyzed through RT-QPCR analysis using gene specific primers for PBEF1. Also in this analysis; therapy-naïve HIV patients displayed significantly higher PBEF1 expression than healthy controls. However, PBEF1 expression in patients on HAART was reduced to the levels also found in healthy controls (FIG. 4). This observation confirms our previous findings and is in accordance with the role of visfatin as an inflammatory product (the viral load in patients on HAART is reduced to undetectable levels, resulting in a decreased inflammatory phenotype). However, it also suggests that circulating monocytes play no role in the increase in plasma visfatin described to occur by Schindler et al in patients undergoing HAART.

### Example 5

### RT-QPCR Analysis of the Effect of HIV Treatment on PBEF1 Expression in Monocyte Samples from Non-Infected Individuals

Monocytes were isolated from buffy coat from healthy control volunteers by counterflow elutriation. These cells were seeded in six-well plates and were treated with infective HIV_{BaL} virus or with virus that had been treated with aldrithiol-2 (AT2), which is reported to covalently modify essential zinc fingers in the nucleocapsid of HIV, rendering it incapable of productive infection while conserving its structure and binding properties (Rossio et al., J. Virol. 1998, 72:7992-8001). Both infective and inactivated viruses were added at a concentration corresponding to 50 ng/ml of p24. RT-QPCR analysis revealed that PBEF1 induction appeared to be an early event in monocyte cultures treated both with active and inactive virus (FIG. 5), suggesting that simple interaction of the virus with the cell is enough to induce significant transcriptional changes in the monocyte.

### Example 6

### Addition of Recombinant Visfatin Decreases the Viral Infectivity of the M-Tropic R5 HIV Labstrain BaL

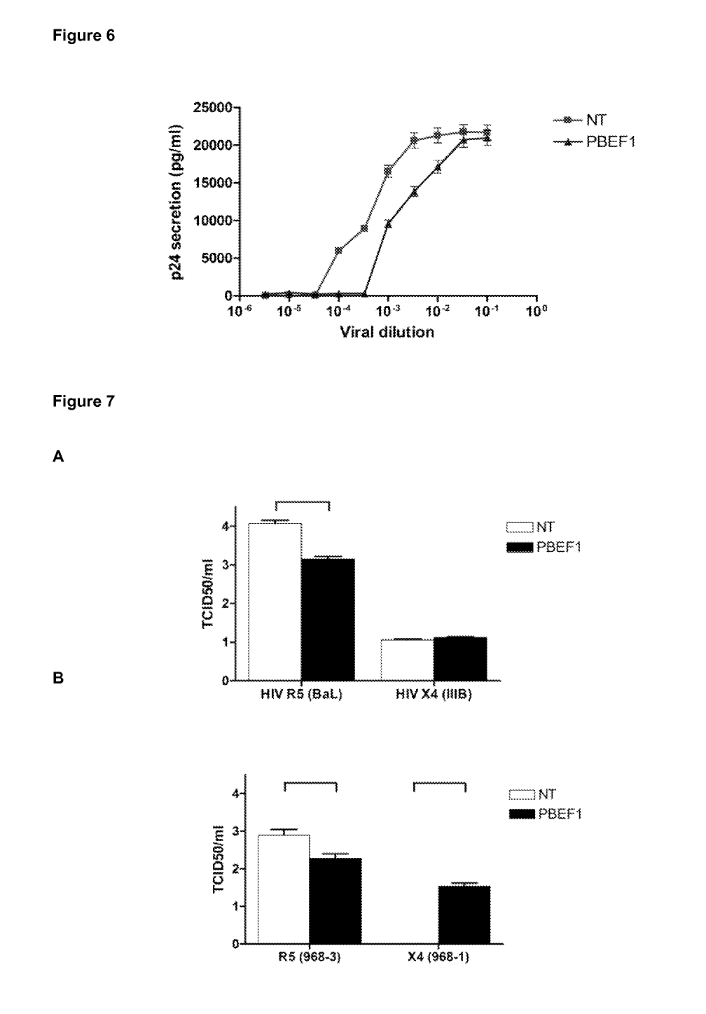

As these expression analyses of visfatin showed interesting tendencies, we initiated a series of experiments aimed at elucidating the functional role of visfatin during HIV infection. Experiments concerning the effect on productive infection with HIV proved to be very promising. Addition of recombinant visfatin to activated PBMC or monocyte-derived macrophage (MDM) cultures prior to in vitro infection with the Mtropic R5 HIV lab-strain BaL decreases the viral infectivity for these cultures (FIG. 6).

This viral infectivity can be quantified by calculating the TCID50 (Tissue Culture Infectious Dose 50%, i.e., the dose of virus that has a 50% chance of infecting a cell culture) values of the viral stocks. Addition of visfatin reduces the TCID50 values of BaL by approximately one log in both PBMC and MDM cultures, signifying a 90% reduction in viral infectivity (FIG. 7, Panel A).

### Example 7

### Visfatin Promotes the HIV Co-Receptor Switch

Interestingly, visfatin does not mediate this effect on the T-tropic X4 HIV lab-strain IIIB (=H×B2). This signifies that by selectively inhibiting R5 and not X4 HIV, visfatin contributes to the HIV co-receptor switch seen in ca. 50% of all patients in which the co-receptor usage of the dominant quasispecies within the patient switches from the moderately virulent R5 to the more aggressive X4, leading to increased progression to AIDS.

Because the BaL and IIIB strains differ significantly in their genetic background and not only in their co-receptor usage, we repeated the same experiments using clinical isolates in lieu of lab strains. R5 and X4 strains of the 968 viral clone were isolated from one patient, and differ only in co-receptor usage. For the R5 strain of the 968 clone (968-3), the results were similar to those of the R5 lab strain BaL. For the X4 strain (986-1), visfatin even increased the viral titer in MDM (FIG. 7, Panel B). While this strain is incapable of infecting untreated MDM, addition of visfatin allowed the 968-1 clone to establish a productive infection.

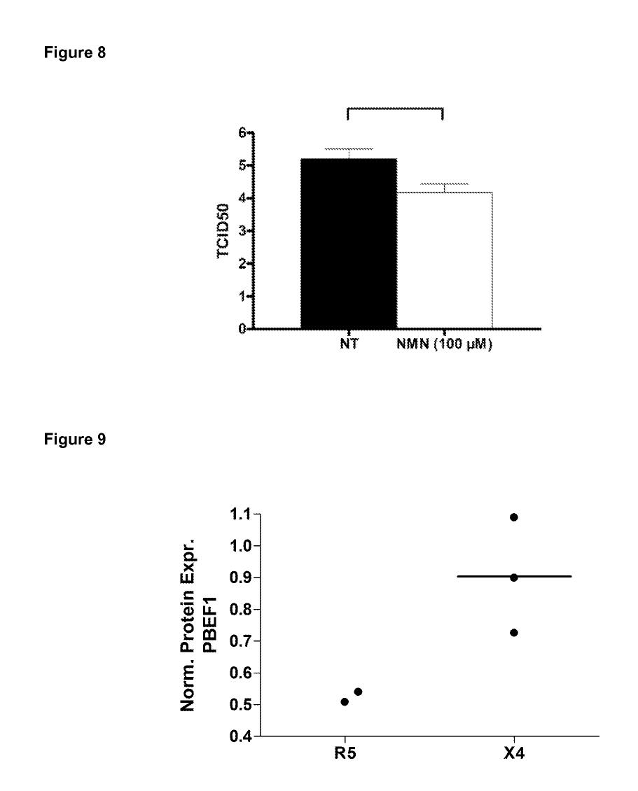

In order to rule out LPS contamination (which also mediates effects on viral infectivity in MDM and PBMC) as a possible contributing factor to this effect, the experiments concerning viral infectivity were reproduced using nicotinamide mononucleotide (NMN), the endproduct of the enzymatic function of visfatin. Simple addition of NMN to PBMC cultures also reduces the infectivity of HIVBaL (FIG. 8), proving that it is, in the first place, the enzymatic function of visfatin that is mediating the described effects, rather than possible LPS contamination of the recombinant protein. Additionally, these results prove that it is specifically the enzymatic function of visfatin that is responsible for these effects, rather than, e.g., the published cytokine-like or insulin-mimetic properties of this molecule.

In order to identify correlates for visfatin expression in HIV patients other than the viral load, several other parameters were examined. One such parameter was the co-receptor, usage of primary viruses isolated from patient plasma samples. Co-receptor usage of these isolates was evaluated through infection experiments of CCR5- or CXCR4-expressing U87 cells (U87.R5 and U87.X4). When patients were grouped according to the co-receptor usage of their corresponding viral isolates (two individuals with R5 virus and three with X4 virus), significant differences were found between the groups at the level of visfatin protein expression. High visfatin expression appeared to correlate with the presence of X4 virus in the clinical isolates, while low visfatin expression was associated with R5 viruses (FIG. 9). This correlation, combined with our observation that visfatin is induced late in infection in patients with high viral loads, supports the application of visfatin as a biomarker for the co-receptor switch, and fits with the contribution of visfatin to that switch.

TABLE 1

| | | | | | | Time |
|---|---|---|---|---|---|---|
| Patient | Age | | | | | infected |
| ID | (years) | Sex | Nationality | T4 | VL | (months) |
| P01 | 30 | M | Belgian | 359 | 3.87 | 8 |
| P02 | 61 | M | Belgian | 748 | 5.54 | 60 |
| P03 | 43 | M | Belgian | 142 | 2.28 | 10 |
| P04 | 33 | M | Subsaharan Africa | 446 | 3.91 | 32 |
| P05 | 39 | M | Cameroon | 644 | 4.34 | 8 |
| P06 | 39 | M | Belgian | 856 | 4.82 | 27 |
| P07 | 36 | M | Belgian | 197 | 5.91 | 48 |
| P08 | 21 | M | Belgian | 133 | <2.70 | 39 |
| P09 | 31 | M | Belgian | 1026 | 3.08 | 9 |
| P10 | 45 | M | Belgian | 781 | 3.50 | 10 |
| P11 | 51 | F | Belgian | 371 | 3.60 | 5 |
| P12 | 43 | M | European | 436 | 4.28 | 48 |
| P13 | 29 | M | Belgian | 532 | 4.78 | 9 |
| P14 | 46 | M | Argentinean | 329 | 5.37 | 4 |
| P15 | 32 | M | Belgian | 738 | 5.58 | 6 |
| P16 | 44 | M | Belgian | 226 | 5.59 | 114 |
| P17 | 45 | M | Belgian | 233 | 5.59 | 36 |
| P18 | 47 | M | Belgian | 359 | 5.84 | 10 |
| P19 | ? | ? | ? | 382 | ND | ? |
| P20 | 39 | M | Belgian | 760 | 3.93 | 66 |
| P21 | 35 | M | Togo | 462 | 4.06 | 17 |
| P22 | 37 | F | Burundi | 374 | 4.24 | 21 |
| P23 | 22 | M | Belgian | 503 | 4.32 | 16 |
| P24 | 48 | M | Belgian | 540 | 4.36 | 27 |
| P25 | 40 | M | Polish | 535 | 4.78 | 20 |
| P26 | 39 | M | Belgian | 746 | 4.90 | 16 |
| P27 | 47 | M | Belgian | 311 | 4.97 | 26 |
| P28 | 39 | M | Belgian | 778 | 5.00 | 8 |
| P29 | 65 | M | Belgian | 756 | 5.07 | 7 |
| C01 | 31 | F | Belgian | ND | NA | NA |
| C02 | 23 | M | Belgian | ND | NA | NA |
| C03 | 23 | F | Belgian | ND | NA | NA |
| C04 | 48 | F | Belgian | ND | NA | NA |
| C05 | 25 | M | Belgian | ND | NA | NA |
| C06 | 22 | F | Belgian | ND | NA | NA |
| C07 | 24 | M | Belgian | ND | NA | NA |
| C08 | 51 | M | Belgian | ND | NA | NA |

Table 1. Clinical details of included patients. T4: CD4+ T lymphocyte count (cells/mm<3>)—ND: not done; VL: viral load (log copies/ml)—NA: not applicable.

TABLE 2

| | Software | | | |
|---|---|---|---|---|
| PPF | application | p-val | % changed | Author |
| Death receptor binding | GenMAPP (GO) | 0.000 | 87.5% | GO |
| FAS signaling cascades. Part 2 | GeneGo | 0.001 | 34.9% | GO |
| TRAF proteins signaling network | GeneGo | 0.001 | 37.1% | GO |
| Role SUMO in p53 regulation | GeneGo | 0.002 | 42.9% | GO |
| AP1 activation by TRAF proteins | GeneGo | 0.002 | 37.9% | GO |
| signaling pathway | | | | |
| Cytoplasm/mitochondrial transport | GeneGo | 0.003 | 34.3% | GO |
| of proapoptotic proteins Bid, Bmf | | | | |
| and Bim | | | | |
| BAD phosphorylation | GeneGo | 0.004 | 26.2% | GO |
| TRADD interaction with MAPK | GeneGo | 0.004 | 34.4% | GO |
| cascade | | | | |
| Caspases activation via nuclear | GeneGo | 0.012 | 33.3% | GO |
| import | | | | |
| Hs Apoptosis | GenMAPP | 0.012 | 38.5% | Alexander C. |
| | (contributed) | | | Zambon and |
| | | | | Beth Lawlor |
| TNFR1 signaling pathway | GeneGo | 0.013 | 28.3% | GO |
| Hs MAPK signaling pathway | GenMAPP | 0.013 | 34.4% | Adapted from |
| KEGG | (contributed) | | | KEGG by |
| | | | | Sebastien |
| | | | | Burel |
| Caspases cascade | GeneGo | 0.021 | 29.4% | GO |
| Apoptosis | GenMAPP (GO) | 0.023 | 32.1% | GO |
| Antiapoptotic Function of | GeneGo | 0.036 | 28.1% | GO |
| TRADD/TRAF2 complex | | | | |
| Hs p38 MAPK signaling pathway | GenMAPP | 0.040 | 41.9% | Adapted from |
| | (contributed) | | | Biocarta by |
| | | | | Sebastien |
| | | | | Burel |
| Role of CARD-protein family in | GeneGo | 0.042 | 30.4% | GO |
| caspase cascade regulation and | | | | |
| apoptosis | | | | |
| p38-MAPK cascade activation via | GeneGo | 0.043 | 28.6% | GO |
| FAS1 and TNFR1 | | | | |
| Caspase cascade activation by | GeneGo | 0.044 | 27.3% | GO |
| FADD and RIPK | | | | |

Table 2. Processes, pathways and molecular functions associated with apoptosis, identified by the software applications GenMAPP and GeneGo as over-represented in CodeLink HWG datasets. PPF: name of the identified process, pathway or function; either the Gene Ontology (GO)/contributed term (GenMAPP) or the name of the curated pathway (GeneGO; on the world-wide web at invitrogen.com/ipath). p-val: p-value of over-represented pathway, as calculated by software application; % changed: percentage of the genes in the pathway that were called as significant; Author: author of contributed genMAPP (on the world-wide web at genmapp.org/).

TABLE 3

| | Software | | | |
|---|---|---|---|---|
| PPF | application | p-val | % changed | Author |
| Hs Insulin Signaling | GenMAPP | 0.000 | 39.6% | Diabetes |
| | (contributed) | | | Genome |
| | | | | Anatomy Project |
| | | | | Investigators |
| Insulin receptor signaling | GenMAPP (GO) | 0.002 | 66.7% | GO |
| pathway | | | | |
| Phospholipid biosynthesis | GenMAPP (GO) | 0.003 | 46.5% | GO |
| Lipid kinase activity | GenMAPP (GO) | 0.014 | 50.0% | GO |
| Phospholipid metabolism | GenMAPP (GO) | 0.016 | 39.7% | GO |
| Membrane lipid metabolism | GenMAPP (GO) | 0.024 | 35.8% | GO |
| Insulin-like growth factor | GenMAPP (GO) | 0.041 | 66.7% | GO |
| receptor binding | | | | |
| Lipid binding | GenMAPP (GO) | 0.041 | 32.7% | GO |
| Membrane lipid biosynthesis | GenMAPP (GO) | 0.042 | 38.2% | GO |

Table 3. Processes, pathways and molecular functions associated with lipid metabolism/insulin resistance, identified by the software applications GenMAPP and GeneGo as over-represented in CodeLink HWG datasets. PPF: name of the identified process, pathway or function; either the Gene Ontology (GO)/contributed term (GenMAPP) or the name of the curated pathway (GeneGO; on the world-wide web at invitrogen.com/ipath). P-val: p-value of over-represented pathway, as calculated by software application; % changed: percentage of the genes in the pathway that were called as significant; Author: author of contributed genMAPP (on the world-wide web at genmapp.org/).

TABLE 4

| | | Gene | | |
|---|---|---|---|---|
| Gene symbol | Name | Entrez ID | Diff Groups | TR |
| ADORA1 | adenosine A1 receptor | 134 | T4 > 200 | I |
| B2M | beta-2-microglobulin | 567 | T4 < 500 | I |
| BCL6 | B-cell CLL/lymphoma 2 | 604 | T4 < 200 | I |
| BNIP2 | BCL2/adenovirus interacting protein 2 | 663 | All | I |
| CAPG | capping protein, gelsolin-like | 822 | All | S |
| CCL18 | chemokine (C-C motif) ligand 18 | 6362 | All | I |
| CCL22 | chemokine (C-C motif) ligand 22 | 6367 | T4 < 500 | S |
| CCL3L1 | chemokine (C-C motif) ligand 3-like 1 | 374793 | T4 < 200 | S |
| CCR1 | chemokine (C-C motif) receptor 1 | 1230 | T4 < 200 | I |
| CCR2_A | chemokine (C-C motif) receptor 2, | 1231 | T4 < 500 | S |
| | isoform A | | | |
| CD83 | CD83 antigen | 9308 | T4 > 500 | I |
| CLEC2D | C-type lectin domain family 2, | 29121 | T4 < 200 | S |
| | member D | | | |
| CNIH2 | cornichon homolog 2 | 254263 | T4 < 500 | I |
| CTNNAL1 | catenin (cadherin-associated), | 8727 | T4 < 200 | I |
| | alpha-like 1 | | | |
| CX3CR1 | chemokine (C—X3—C motif) receptor 1 | 1524 | T4 < 500 | S |
| CXCL2 | chemokine (C—X—C motif) ligand 2 | 2920 | All | S, I |
| DDIT3 | DNA-damage-inducible transcript 3 | 1649 | All | I |
| EN2 | engrailed homolog 2 | 2020 | T4 < 500 | I |
| IL1A | interleukin 1, alpha | 3552 | All | I |
| IL1B | interleukin 1, beta | 3553 | T4 > 500 | I |
| LAMP2_2B | lysosomal-associated membrane | 3920 | All | I |
| | protein 2, isoform 2B | | | |
| LAT | linker for activation of T cells | 27040 | T4 > 200 | S |
| LILRB4 | leukocyte immunoglobulin-like | 11006 | All | S |
| | receptor, B4 | | | |
| LOC374794 | onbekend | 374794 | All | S, I |
| LTB4DH | leukotriene B4 | 22949 | T4 < 500 | I |
| | 12-hydroxydehydrogenase | | | |
| MAPK10 | mitogen-activated protein kinase 10 | 5602 | T4 > 500 | I |
| MRC1 | mannose receptor, C type 1 | 4360 | T4 < 500 | I |
| PBEF1 | pre-B-cell colony-enhancing factor 1 | 10135 | All | I |
| PCDH7_b | BH-protocadherin, isoform b | 5099 | T4 > 500 | I |
| PLA2G7 | phospholipase A2, group VII | 7941 | All | S |
| PTGER2 | prostaglandin E receptor 2 | 5732 | T4 < 500 | I |
| STAT1_a | signal transducer and activator of | 6772 | T4 < 500 | I |
| | transcription 1, isoform a | | | |
| STAT1_b | signal transducer and activator of | 6772 | T4 < 500 | I |
| | transcription 1, isoform b | | | |
| TEBP | unactive progesterone receptor | 10728 | T4 < 500 | I |
| TIEG | TGFB inducible early growth | 7071 | T4 < 500 | I |
| | response | | | |
| TNFAIP3 | tumor necrosis factor alpha-induced | 7128 | T4 > 500 | I |
| | protein 3 | | | |
| XLKD1 | extracellular link domain containing 1 | 10894 | T4 < 200 | I |
| YWHAZ | tyrosine 3-monooxygenase activation | 7534 | T4 < 200 | I |
| | protein | | | |

Table 4. Genes identified from the MAS analysis as differentially expressed between samples from HIV patients and controls. T4: CD4+ T lymphocyte count (cells/mm<3>). Diff Groups: Groups of patients (defined by T4 counts) in which the genes are differentially expressed. TR: type of regulation: I=induction, S=suppression.

TABLE 5

| | | Entrez | | | | |
|---|---|---|---|---|---|---|
| Gene name | Symbol | ID | Group | Evidence | FC | P-val |
| adenosine A1 receptor | ADORA1 | 134 | All | GO | −1.63 | 0.045 |
| pre-B-cell | PBEF1 | 10135 | All | Jia et al., | 2.74 | 0.003 |
| colony-enhancing | | | | 2004 | | |
| factor 1 | | | | | | |
| tumor necrosis factor | TNFAIP3 | 7128 | >500 | GO | 2.21 | 0.030 |
| alpha-induced protein 3 | | | | | | |
| signal transducer and | | | | | | |
| activator of | | | | | | |
| transcription 1 | | | | | | |
| isoform alpha | STAT1 (α) | 6772 | All | GO | 1.88 | 0.007 |
| isoform beta | STAT1 (β) | 6772 | All | GO | 1.57 | 0.006 |
| DNA-damage-inducible | DDIT3 | 1649 | <200 | Oyadomari | 1.67 | 0.018 |
| transcript 3 | | | | & Mori, | | |
| | | | | 2004 | | |
| BCL2/adenovirus E1B | BNIP2 | 663 | >500 | GO | 1.55 | 0.001 |
| 19 kDa interacting | | | | | | |
| protein 2 | | | | | | |

Table 5. Apoptosis-associated genes differentially expressed between monocytes of HIV patients and of healthy controls, as assessed by custom MAS array analysis. Group: patient group, based on CD4+ T lymphocyte count (cells/mm<3>); Evidence: evidence for assigning the gene to the cluster “Apoptosis-associated genes”; GO: Gene Ontology annotation; FC: fold change; P-val: p-value, determined via uncorrected student's t-test.

TABLE 6

| | Viral | | | | | |
|---|---|---|---|---|---|---|
| | load | T4 | | | | |
| | (log | count | | | | Months |
| | copies/ | (cells/ | | | Months | on |
| ID | ml) | μl) | Age | Nationality | infected | therapy |
| HAART001 | 0.00 | 352 | 33 | Belgian | 3 | 12 |
| HAART002 | 0.00 | 430 | 71 | Dutch | 25 | 21 |
| HAART003 | 0.00 | 468 | 33 | Belgian | 42 | 23 |
| HAART004 | 0.00 | 328 | 37 | Belgian | 112 | 23 |
| HAART005 | 0.00 | 416 | 52 | Belgian | 40 | 25 |
| HAART006 | 0.00 | 781 | 38 | Sub-Saharan | 73 | 64 |
| | | | | Africa | | |
| HIV001 | 5.8 | 464 | 48 | Belgian | 8 | N.A. |
| HIV002 | 5.08 | 874 | 26 | Nigerian | 13 | N.A. |
| HIV003 | 5.60 | 775 | 41 | Central | 35 | N.A. |
| | | | | American | | |
| HIV004 | 4.97 | 365 | 43 | Belgian | 40 | N.A. |
| HIV005 | 4.37 | 576 | 41 | Belgian | 41 | N.A. |
| HIV006 | 5.49 | 312 | 34 | Belgian | 45 | N.A. |
| HIV007 | 4.94 | 295 | 42 | Belgian | 73 | N.A. |
| C001 | N.A. | N.A. | 52 | Belgian | N.A. | N.A. |
| C002 | N.A. | N.A. | 28 | Kenian | N.A. | N.A. |
| C003 | N.A. | N.A. | 34 | Belgian | N.A. | N.A. |
| C004 | N.A. | N.A. | 31 | Belgian | N.A. | N.A. |
| C005 | N.A. | N.A. | 54 | Belgian | N.A. | N.A. |

Table 6. Clinical details of patients enrolled in the study for PBEF1 levels in the monocytes of therapy-naïve patients and patients on HAART. T4: CD4+ T lymphocyte count (cells/mm<3>); NA: not applicable.

### REFERENCES

Abbate I., F. Dianzani, and M. R. Capobianchi (2000). Activation of signal transduction and apoptosis in healthy lymphocytes exposed to bystander HIV-1-infected cells. Clin. Exp. Immunol. 122:374-380.
Aboud M., A. Elgalib, R. Kulasegaram, and B. Peters (2007). Insulin resistance and HIV infection: a review. Int. J. Clin. Pract. 61:463-472.
Anderson E., W. Zink, H. Xiong, and H. E. Gendelman (2002). HIV-1-associated dementia: a metabolic encephalopathy perpetrated by virus-infected and immune-competent mononuclear phagocytes. J. Acquir. Immune Defic. Syndr. 31:S43-S54.
Aquaro S., R. Caliò, J. Balzarini, M. C. Bellocchi, E. Garaci, and C. F. Perno (2002). Macrophages and HIV infection: therapeutical approaches toward this strategic virus reservoir. Antiviral Res. 55:209-225.
Asensio V. C., J. Maier, R. Milner, K. Boztug, C. Kincaid, M. Moulard, C. Phillipson, K. Lindsley, T. Krucker, H. S. Fox, and I. L. Campbell (2001). Interferon-independent, human immunodeficiency virus type 1 gp120-mediated induction of CXCL10/IP-10 gene expression by astrocytes in vivo and in vitro. J. Virol. 75:7067-7077.
Ashburner M., C. A. Ball, J. A. Blake, D. Botstein, H. Butler, J. M. Cherry, A. P. Davis, K. Dolinski, S. S. Dwight, J. T. Eppig, M. A. Harris, D. P. Hill, L. Issel-Tarver, A. Kasarskis, S. Lewis, J. C. Matese, J. E. Richardson, M. Ringwald, G. M. Rubin, and G. Sherlock (2000). Gene ontology: tool for the unification of biology. The Gene Ontology Consortium. Nat. Genet. 25:25-29.
Benjamini Y. and Y. Hochberg (1995). Controlling the false discovery rate: a practical and powerful approach to multiple testing. J. Roy. Stat. Soc. B. 57:289-300.
Beirnaert E., B. Willems, M. Peeters, A. Bouckaert, L. Heyndrickx, P. Zhong, S. Vereecken, D. Coppens, D. Davis, P. Ndumbe, W. Janssens, and G. van der Groen (1998). Design and evaluation of an in-house HIV-1 (group M and O) SIVmnd and SIVcpz antigen capture assay. J. Virol. Methods 73:65-70.
Doniger S. W., N. Salomonis, K. D. Dahlquist, K. Vranizan, S. C. Lawlor, and B. R. Conklin (2003). MAPPFinder: using Gene Ontology and GenMAPP to create a global gene-expression profile from microarray data. Genome Biol. 4:R7.
Federico M., Z. Percario, E. Olivetta, G. Fiorucci, C. Muratori, A. Mitchell, G. Romeo and E. Affabris (2001). HIV-1 Nef activates STAT1 in human monocytes/macrophages through the release of soluble factors. Blood 98:2752-2761.
Freedman B. D., Q. H. Liu, M. Del Corno and R. G. Collman (2003). HIV-1 gp120 chemokine receptor-mediated signaling in human macrophages. Immunol. Res. 27:261-276.
Izmailova E., F. M. Bertley, Q. Huang, N. Makori, C. J. Miller, R. A. Young, and A. Aldovini (2003). HIV-1 Tat reprograms immature dendritic cells to express chemoattractants for activated T cells and macrophages. Nat. Med. 9:191-197.
Jia S. H., Y. Li, J. Parodo, A. Kapus, L. Fan, O. D. Rotstein, and J. C. Marshall (2004). Pre-B cell colony-enhancing factor inhibits neutrophil apoptosis in experimental inflammation and clinical sepsis. J. Clin. Invest. 113:1318-1327.
Kedzierska K., R. Azzam, P. Ellery, J. Mak, A. Jaworowski, and S. M. Crowe (2003). Defective phagocytosis by human monocyte/macrophages following HIV-1 infection: underlying mechanisms and modulation by adjunctive cytokine therapy. J. Clin. Virol. 26:247-263.
Mahlknecht U. and G. Herbein (2001). Macrophages and T-cell apoptosis in HIV infection: a leading role for accessory cells? Trends Immunol. 22:256-260.
Moschen A. R., A. Kaser, B. Enrich, B. Mosheimer, M. Theurl, H. Niederegger, and H. Tilg (2007). Visfatin, an adipocytokine with proinflammatory and immunomodulating properties. J. Immunol. 178:1748-1758.
Ognjanovic S., T. L. Ku, and G. D. Bryant-Greenwood (2005). Pre-B-cell colony-enhancing factor is a secreted cytokine-like protein from the human amniotic epithelium. Am. J. Obstet. Gynecol. 193:273-282.
Oyadomari S. and M. Mori (2004). Roles of CHOP/GADD153 in endoplasmic reticulum stress. Cell Death Differ. 11:381-389.
Roberts E. S., M. A. Zandonatti, D. D. Watry, L. J. Madden, S. J. Henriksen, M. A. Taffe, and H. S. Fox (2003). Induction of pathogenic sets of genes in macrophages and neurons in NeuroAIDS. Am. J. Pathol. 162:2041-2057.
Rossio J. L. M. T. Esser, K. Suryanarayana, D. K. Schneider, J. W. Bess, G. M. Vasquez, T. A. Wiltrout, E. Chertova, M. K. Grimes, Q. Sattentau, L. O. Arthur, L. E. Henderson, and J. D. Lifson (1998). Inactivation of human immunodeficiency virus type 1 infectivity with preservation of conformational and functional integrity of virion surface proteins. J. Virol. 72:7992-8001.
Samal B., Y. Sun, G. Stearns, C. Xie, S. Suggs, and I. McNiece (1994). Cloning and characterization of the cDNA encoding a novel human pre-B-cell colony-enhancing factor. Mol. Cell. Biol. 14:1431-1437.
Schindler K., D. Haider, M. Wolzt, A. Rieger, B. Gmeinhart, A. Luger, P. Nowotny, and B. Ludvik (2006). Impact of antiretroviral therapy on visfatin and retinol-binding protein 4 in HIV-infected subjects. Eur. J. Clin. Invest. 36:640-646.
Stephens J. M. and A. J. Vidal-Puig (2006). An update on visfatin/pre-B cell colony-enhancing factor, a ubiquitously expressed, illusive cytokine that is regulated in obesity. Curr. Opin. Lipidol. 17:128-131.
Swingler S., A. Mann, J. Jacque, B. Brichacek, V. G. Sasseville, K. Williams, A. A. Lackner, E. N. Janoff, R. Wang, D. Fisher and M. Stevenson (1999). HIV-1 Nef mediates lymphocyte chemotaxis and activation by infected macrophages. Nat. Med. 5:997-1003.
Swingler S., B. Brichacek, J. M. Jacque, C. Ulich, J. Zhou and M. Stevenson (2003). HIV-1 Nef intersects the macrophage CD40L signaling pathway to promote resting-cell infection. Nature 424:213-219.
Van den Bergh R., G. Vanham, G. Raes, P. De Baetselier, and R. Colebunders (2006). Mycobacterium-associated immune reconstitution disease: macrophage running wild? Lancet Infect. Dis. 6:2-3.—van der Veer E., Z. Nong, C. O'Neil, B. Urquhart, D. Freeman, and J. G. Pickering (2005). Pre-B-cell colony-enhancing factor regulates NAD+-dependent protein deacetylase activity and promotes vascular smooth muscle cell maturation. Circ. Res. 97:24-34.
Van Herrewege Y., L. Penne, C. Vereecken, K. Fransen, G. van der Groen, L. Kestens, J. Balzarini, and G. Vanham (2002). Activity of reverse transcriptase inhibitors in monocyte-derived dendritic cells: a possible in vitro model for postexposure prophylaxis of sexual HIV transmission. AIDS Res. Hum. Retrovi. 18:1091-1102.

1. A method for diagnosis, prognosis or theranosis of an HIV-related disease, the method comprising:
(a) collection of a blood sample from a subject,
(b) isolation of the monocytes from this blood sample, and
(c) determination of gene expression in said monocytes.

2. The method according to claim 1, wherein said gene expression is PBEF1 mRNA expression.

3. The method according to claim 1, wherein said gene expression is the measurement of PBEF1 protein.

4. The method according to claim 1, wherein said HIV-related disease is selected from the group consisting of Acquired Immune Deficiency Syndrome, HIV-associated dementia, Immune Reconstitution Disease, and lipodystrophy.

5. The method according to claim 2, wherein the HIV-related disease is selected from the group consisting of Acquired Immune Deficiency Syndrome, HIV-associated dementia, Immune Reconstitution Disease, and lipodystrophy.

6. The method according to claim 1, wherein the HIV-related disease is selected from the group consisting of Acquired Immune Deficiency Syndrome, HIV-associated dementia, Immune Reconstitution Disease, and lipodystrophy.

7. A method for monitoring the progression of an HIV-related disease, the method comprising:
collecting a blood sample from a subject diagnosed as suffering from an HIV-related disease,
isolating of the monocytes from the blood sample, and
determining PBEF1 gene expression in the isolated monocytes.

8. The method according to claim 7, wherein PBEF1 gene expression is determined by measuring PBEF1 mRNA expression.

9. The method according to claim 7, wherein PBEF1 gene expression is determined by measuring PBEF1 protein production.

10. The method according to claim 8, wherein PBEF1 gene expression is determined by measuring PBEF1 protein production.

11. The method according to claim 9, wherein PBEF1 gene expression is determined by measuring PBEF1 mRNA expression.

12. The method according to claim 7, wherein the HIV-related disease is selected from the group consisting of Acquired Immune Deficiency Syndrome, HIV-associated dementia, Immune Reconstitution Disease, and lipodystrophy.

13. The method according to claim 8, wherein the HIV-related disease is selected from the group consisting of Acquired Immune Deficiency Syndrome, HIV-associated dementia, Immune Reconstitution Disease, and lipodystrophy.

14. The method according to claim 9, wherein the HIV-related disease is selected from the group consisting of Acquired Immune Deficiency Syndrome, HIV-associated dementia, Immune Reconstitution Disease, and lipodystrophy.

15. The method according to claim 10, wherein the HIV-related disease is selected from the group consisting of Acquired Immune Deficiency Syndrome, HIV-associated dementia, Immune Reconstitution Disease, and lipodystrophy.

16. The method according to claim 11, wherein the HIV-related disease is selected from the group consisting of Acquired Immune Deficiency Syndrome, HIV-associated dementia, Immune Reconstitution Disease, and lipodystrophy.


SEQUENCE LISTING (4 sequences)

SEQ ID NO: 1 - DNA, 20 bp, Artificial Sequence
  Gene specific primers for the gene of interest (PBEF1.F)
ggcaaggtgacaaaaagcta

SEQ ID NO: 2 - DNA, 20 bp, Artificial Sequence
  Gene specific primers for the gene of interest (PBEF1.R)
atgaaagggcagtatgtcca

SEQ ID NO: 3 - DNA, 21 bp, Artificial Sequence
  Housekeeping gene (GAPDH.F)
agctcatttcctggtatgaca

SEQ ID NO: 4 - DNA, 20 bp, Artificial Sequence
  Housekeeping gene (GAPDH.R)
tggttgagcacagggtactt